(19) United States
(12) Patent Application Publication
(10) Pub. No.: US 2024/0268919 A1
(43) Pub. Date: Aug. 15, 2024

(54) ROBOTICALLY COORDINATED SURGICAL VISUALIZATION

(71) Applicant: LEM Surgical AG, Bern (CH)

(72) Inventor: Yossi Bar, Bern (CH)

(73) Assignee: LEM Surgical AG, Bern (CH)

(21) Appl. No.: 18/628,142

(22) Filed: Apr. 5, 2024

Related U.S. Application Data
(63) Continuation-in-part of application No. PCT/IB2022/058986, filed on Sep. 22, 2022.
(60) Provisional application No. 63/270,487, filed on Oct. 21, 2021.

(51) Int. Cl. A61B 90/00; A61B 34/32; A61B 90/25

(52) U.S. Cl. CPC A61B 90/37; A61B 34/32; A61B 90/25; A61B 90/361; A61B 2090/365; A61B 2090/368

(57) ABSTRACT

Robotically controlled and coordinated surgical navigation systems include displays which may have virtual and/or augmented reality capabilities. Multi-arm robotic systems hold cameras, tools, and virtual and/or augmented reality screens The robotic arms are deployed on a chassis incorporating a control unit. Multiple robotic elements may be attached to the single base and may be controlled by the single control unit in order to be used in a coordinated fashion to deploy and/or relate to trackers, cameras, virtual and/or augmented reality screens and surgical instruments as part of a robotic surgery procedure that may optionally be a spinal robotic surgery procedure.

### CROSS-REFERENCE TO RELATED APPLICATIONS

This application is a continuation-in-part of pending international application PCT/IB2022/058986, filed on Sep. 22, 2022, which claims the benefit of U.S. Provisional Patent Application 63/270,487, filed on Oct. 21, 2021, the full disclosures of each of which are incorporated herein by reference in their entirety.

### BACKGROUND

Field. The disclosed technology relates generally to medical systems and methods. More particularly, the disclosed technology relates to surgical robots and methods and to apparatus for augmenting actual and virtual visualization of a robotic workspace.

Robotic surgery refers to the application of robotic techniques to a variety of surgeries, including but not limited to prostate and other urologic surgeries, cardiac bypass and other coronary procedures, gynecological surgeries, spinal and other orthopedic surgeries. The Sapien™ surgical robot is being developed by LEM Surgical AG, the assignees of the present application, for spinal and other orthopedic surgeries. The Sapien™ surgical robot is a multi-armed robot mounted on a mobile cart configured to be placed beneath a surgical table to locate one or more tool-bearing arms on opposite sides of the table. The basic structure and function of the Sapien™ surgical robot are described in PCT application no. PCT/IB2022/052297 (published as WO2022/195460), commonly owned with the present application, the full disclosure of which is incorporated herein by reference.

Enhanced imaging techniques, such as virtual reality and/or augmented reality (VR/AR) capabilities, have been incorporated into surgery in general and robotic surgery in particular. The use of VR/AR and other enhanced imaging tools has been suggested, for example, to help users navigate surgical tools through anatomies which lack direct line of sight and where it is difficult the to the tool orient and/or distinguish anatomical features. Image guidance and visualization can be based, in whole or in part, on preoperative images and intraoperative imaging, including but not limited to computed tomography (CT) imaging, magnetic resonance imaging (MRI), X-ray imaging, fluoroscopic imaging, ultrasound imaging, and the like. In some cases, a tracked tool position can be superimposed on a preoperative or real-time anatomical image which can be shown on a display or projected onto a patient's skin.

Current VR/AR and other enhanced imaging systems intended for robotic surgery, however, have significant drawbacks. For example, available and proposed enhanced surgical robotic imaging systems are often passive, e.g. a display or other system component must be positioned and repositioned in the surgical field by the user during the robotic surgery. VR/AR and other imaging capabilities are often poorly integrated in surgical robots, typically be added onto a navigation or other pre-existing camera. As navigation camera are often distant from the surgical field, the quality and alignment of the virtual and actual images can be compromised. Many present VR/AR displays are inconvenient to deploy and difficult to watch.

As an alternative to the use of external display screens, Beyeonics Vision Ltd., Haifa, Israel, has developed a headset which can be worn by a user and which provides real time imaging when used with a robotically supported camera and/or microscope. A single camera and/or microscope is mounted on a dedicated robotic arm on a mobile cart that can be positioned by the user. The system is described in WO2020/084625. The need to wear a headset can be limiting to many users.

Accordingly, there is a need for alternative and additional imaging and display capabilities suitable for use with surgical robots and robotic surgeries. In particular, it would be desirable to provide surgical robotic imaging and display capabilities that can be automatically controlled and coordinated by the surgical robot to reduce or minimize user input and distraction during a robotic surgical procedure. It would be particularly desirable if the imaging and display capabilities were at least partially integrated with existing robotic technology and/or navigation technology to facilitate implementation and use. For example, it would be advantageous to have cameras and other imaging instruments as well as display screens manipulated by a common robotic controller to optimize views and information presented to the user. At least some of these objectives will be met by the technology disclosed and claimed herein.

Listing of Background Art. WO2022/195460 and WO2020/084625 has been described above. Other commonly owned patent publications include PCT application no. PCT/IB2022/058986 (published as WO2023/067415); PCT application no. PCT/IB2022/058972 (published as WO2023/118984); PCT application no. PCT/IB2022/058982 (published as WO2023/118985); PCT application no. PCT/IB2022/058978 (published as WO2023/144602); PCT application no. PCT/IB2022/058980 (published as WO2023/152561); PCT application no. PCT/IB2023/055047 (published as WO2023/223215); PCT application no. PCT/IB2022/058988 (published as WO2023/237922); PCT application no. PCT/IB2023/055439; PCT application no. PCT/IB2023/056911; PCT application no. PCT/IB2023/055662; PCT application no. PCT/IB2023/055663; U.S. provisional application no. 63/524,911; and U.S. provisional application no. 63/532,753, the full disclosures of each of which are incorporated herein by reference. Other patent publications of interest include US2019/0088162; US2021/093404; US2021/289188; and U.S. Pat No. 9,918,681.

### SUMMARY

In a first aspect, the disclosed technology provides a surgical robotic system comprising a chassis. A first surgical robotic arm can be mounted on the chassis and is configured to carry an imaging device, typically a camera, a microscope, or other sensor configured to provide locational or other information regarding a target anatomy of a patient undergoing a robotic surgical procedure, e.g., a vertebra or other bony structure of a patient undergoing an orthopedic procedure. A second surgical robotic arm can be mounted on the chassis and configured to carry a display screen, and a third surgical robotic arm mounted on the chassis and is configured to carry a surgical tool. A robotic controller, typically but not necessarily mounted on the chassis, can be configured to (1) control movement of the first surgical robotic arm to position the imaging device to view the target anatomy, (2) control movement of the second surgical robotic arm to orient the display screen in a predetermined spatial relationship with the target location on the patient anatomy, and (3) display an image of the patient's anatomy on the display screen.

The display screen, the imaging device, and the surgical tool may each be permanently affixed or detachably connected to the associated surgical robotic arm. Typically, at least the surgical tool(s) can be detachably connected to the associated surgical robotic arm(s), allowing the tools and other end effectors to be interchanged for use in different robotic surgical procedures. Often, but not always, the display screen and the imaging device can be permanently affixed to the associated surgical robot arm as these robotic components will be used in many or all surgical robotic protocols and require special electronic and/or mechanical connections that may not be available on “working” surgical robotic arms intended for interchangeably deploying a variety of specific surgical tools.

The display can be configured to show any one or more of a variety of images. Most often, the images can comprise, or be derived from, preoperative images including but not limited to computed tomography (CT) images, magnetic resonance images (MRI), X-ray images, fluoroscopic images, ultrasound images, and the like. Usually, the preoperative images can show internal anatomy which is not externally visible, such as bony structure useful in performing robotic spinal and other orthopedic procedures. Other internal anatomies that can be shown include the patient's vasculature, body organs, and the like.

In other instances, the images can comprise real-time images being acquired by the system imaging device or another imaging device, such as a surgical microscope. In such instances, the display can often provide an enlarged or otherwise enhanced image unavailable to a user by direct viewing. The disclosed technology can automatically or manually allow positioning of the display screen so that it is in alignment with the target anatomy or otherwise positioned for maximum use.

As used herein and in the claims, “automatic” and “automatically” can mean that the controller will position a surgical robotic arm or control a tool or the display screen based upon an algorithm or protocol with minimal or no input from the user. In contrast, the terms “manual” and “manually” can mean that the user effects positioning of the surgical robotic arm or control of the tool or the display screen either directly, e.g., physically grasping and moving a tool/robot arm assembly, or indirectly by instructing the controller to make a specific move or system adjustment using an interface with the controller, e.g., a touch screen, a track pad, a mouse, a joystick, a roller ball, and the like, typically but not necessarily mounted on the chassis.

In some instances, the controller will automatically position the display screen along a user's line-of-sight. In such cases, the display screen may be at least partly transparent and configured to allow a user to view an image on the display while maintaining a line-of-sight view of the target anatomy through the display screen. This configuration is particularly useful when the display screen is showing internal patient anatomy which cannot be seen by direct vision or external optical cameras.

In some instances, the display screen can be positioned over the target anatomy and the anatomical display image can include internal structures not externally visible, e.g., bone structure in spinal and other orthopedic robotic surgeries.

In these instances, the display screen may be located over the surgical tool, allowing a user to align the tool with a target internal anatomical structure visible on the anatomical display image. Such tool alignment can normally be effected through a user interface on the surgical controller, allowing the user to manipulate the tools as the user watches the tool and the display. Suitable user interfaces can comprise a touch screen, a track pad, a mouse, a joystick, a roller ball, and the like.

In some instances, the imaging device may be a surgical microscope and the controller may be configured to display an output of the surgical microscope on the display screen while the display screen and the target location are in the user's line-of-site.

In some instances, the controller may be configured to (1) at least in part automatically control the first and second robot surgical arms and (2) allow a user to at least in part control the third surgical robotic arm in real-time. As noted above, such real-time control may be effected by the user employing an inter robotic controller interface.

In some instances, the display screen may itself include an imaging device in addition to the imaging device carried by the first surgical robotic arm. While the imaging device carried by the first surgical robotic arm can be a three-dimensional navigation camera, the additional imaging device on the display may be a smaller device, such as a CCD camera useful to allow the controller to optically track the location of the display screen relative to the patient, the surgical tool, and other robotic components.

In some instances, the controller may be configured to align the display screen with the patient anatomy based at least in part on an output of the additional image sensor.

In some instances, the controller is further configured to receive position information showing the location of the user's eyes relative to the display, typically with reference to the robotic surgical coordinate space.

In some instances, the controller may be further configured to determine a line-of-sight from the position of the user's eyes to the target location.

In a second aspect, the disclosed technology provides a method for performing robotic surgery on a patient, The method comprises controlling a first surgical robotic arm to position an imaging sensor to scan a target surgical site on a patient anatomy, controlling a second surgical robotic arm to orient a display screen in a predetermined relationship with the target surgical site on the patient anatomy, and controlling a third surgical robotic arm to position a surgical tool to be used in performing the robotic surgery. A user is positioned adjacent to the patient at a location which allows the user to direct view both the display screen and the target location. The first and second surgical robotic arms are at least in part automatically controlled by a robotic controller, and the third surgical robotic arm is at least in part controlled by real-time input from the user to the controller as the user views the display and the patient anatomy. As noted previously, such real-time input is typically input through a robotic controller interface.

In some instances, a preoperative image is displayed on the display screen.

In some instances, a real-time image is displayed on the display screen.

In some instances, the display screen is at least partly transparent allowing the user to view a preoperative or other image on the display, typically showing bone or other internal anatomical structure, while maintaining a line-of-sight view of the surgical site through the display screen.

In some instances, the robotic controller controls (usually automatically without direct user input) at least the first surgical robotic arm to align the display screen along a line-of-sight from the user's eyes to the surgical site on the patient's anatomy. For example, the controller may control a camera or other sensor to scan the user to determine a position of the user's eyes. In some cases, the controller could use the imaging device carried by the first surgical robotic arm to scan the user's eyes.

In some instances, the imaging sensor may comprise a surgical microscope and the robotic controller may deliver an image from the microscope to the display screen and automatically or otherwise position the display screen in line-of-sight with the patient anatomy being viewed by the surgical microscope.

In still further aspects, the technologies disclosed herein provide to robotically controlled and coordinated surgical navigation systems that may include virtual and/or augmented reality capabilities. In particular, the disclosed technology relates to navigation systems wherein multiple robotic elements, such as robotic arms, end effectors, surgical instruments, cameras, imaging devices, tracking devices, virtual and/or augmented reality screens or other devices useful for robotic surgery are incorporated and wherein the placement and movement of the robotic elements are controlled and coordinated by a single control unit, and wherein all of the robotic elements are based on a single mobile rigid chassis and, thus, are robotically coordinated at a single origin point. Specifically, multiple robotic elements may be attached to, and controlled by, a single control unit and may be used in a coordinated fashion to deploy and/or relate to trackers, cameras, virtual and/or augmented reality screens and surgical instruments as part of a robotic surgery procedure. More particularly, in the context of robotic spinal surgery, multiple robotic elements may be attached to, and controlled by, a single control unit and may be used in a centrally coordinated fashion to deploy trackers, hold one or more cameras and/or virtual/virtual and/or augmented reality screens, and carry out a surgical procedure, with the relative movements of each robotic element being coordinated by the central control unit. The virtual and/or augmented reality elements provided herein are active from the perspective of the user's view.

Provided herein is a robotically controlled surgical navigation system. Specifically provided herein is a robotically controlled surgical navigation system for robotic orthopedic and spinal surgery with virtual and/or augmented reality capabilities. The system is configured to perform all aspects of robotic spinal/orthopedic surgery procedures, beyond simple steps such as pedicle screw placement that are performed by currently known robotic systems. The system is further configured to provide enhanced visibility and capabilities in surgical procedures through the use of virtual and/or augmented reality capabilities.

In representative embodiments, the system comprises a central control unit housed by a mobile surgical cart. At least two arms or other holders may be mounted to the cart. The at least two arms or holders are configured to hold cameras, sensors, virtual and/or augmented reality screens, end effectors or other instruments to be used in surgery, more particularly in spinal surgery. The at least two arms or holders may also be used to track passive or active markers in the surgical field, that are attached to soft or hard tissue, particularly to spinal bones, wherein the markers have usually been deployed by the physician or user near the beginning of the surgical procedure. Active or passive markers may also optionally be attached to various relevant surfaces, such as the surgical table, surgical poles or stands and also the arms or holders and also to the virtual/augmented reality screen themselves. The disclosed robotically coordinated surgical system provides that the arms or other holders are centrally coordinated by the control unit housed in the surgical cart. Solely by way of example, this allows one arm or holder to be deploying a surgical instrument in relation to a bone marker or a specific element in space while another arm or holder deploys a navigation/tracking camera or virtual and/or augmented reality component at an appropriate distance and angulation, all of which allows for coordinated deployment of surgical instruments, navigation components and all may be presented in a coordinated fashion in the virtual/augmented reality screens which are also robotically held and coordinated.

In various embodiments, passive or active markers may be used to assist in navigation during a surgical procedure, and in particular during a spinal surgery procedure. Spinal surgery procedures may require the placement of multiple passive or active markers on the bony anatomy of multiple vertebrae or in combination with additional markers on the skin, surgical table, stands etc. In particular embodiments, miniature markers may be preferred e.g., smaller than 5 cm. Moreover, vertebrae are relatively small and so to place multiple markers on one or more vertebrae it may be advantageous to use relatively small markers (1 cm or less in size). When using small markers, it may be advantageous to have the one or more cameras/sensors be deployed quite close to the surgical field, for example at a distance of 30 cm or less from the surgical field, and also at an advantageous angulation relative to the surgical field so that the marker(s) can be visualized. For example, if a small marker is deployed at an inconvenient angle inside the patient's body, it will be advantageous to position the camera at a close distance and an appropriate angle. This is also true for a virtual or augmented reality device.

In various embodiments of the disclosed technology, active/robotic virtual and/or augmented reality features are provided to the user. In these embodiments, at least one of the centrally coordinated robotic arms may hold a virtual and/or augmented reality screen. The virtual and/or augmented reality screen is actively brought to the surgical field by the robotic arm that it is mounted on and the arm knows where to bring the virtual and/or augmented reality screen based on the centrally coordinated guidance controlled from the central chassis. Optionally, a navigation camera may be integrated into the virtual and/or augmented reality screen such that the camera is providing location information and feedback to the central control unit on the chassis that then provides feedback information to the robotic arm carrying the virtual and/or augmented reality screen (and integrated camera) and actively guides its motion. Thus, for example, if an active or passive marker has been placed on the anatomy by the user at the beginning or during the surgical procedure, the robotic central control unit can “tell” the robotic arm carrying the virtual and/or augmented reality screen and integrated camera to move toward the marker and the camera is able to confirm that the virtual and/or augmented reality screen has reached the correct location, at which point it can be used to provide the user with additional guidance. It is emphasized that the same method can be performed without the added navigation camera on the screen since all robotic arms are robotically coordinated and synchronized with one controller, so all robotic motion, including the motion of the robotic arm which holds the screen, is by definition robotically coordinated. The addition of the navigation close loop is an additional safety layer that may or may not be used in this process.

All of these needs and elements benefit tremendously from the central coordination and control of the disclosed single-cart, multi-arm, non-teleoperated robotic system.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a surgical robotic system constructed in accordance with some embodiments.

FIGS. 2A and 2B are isolated views showing a user's view of a surgical tool with and without use of a “head-up” display, in accordance with some embodiments.

FIG. 3 illustrates robotic alignment of a display screen with a user's field-of-view of a patient on a surgical table, in accordance with some embodiments.

FIG. 4 illustrates a surgical robotic system constructed in accordance with the principles of the disclosed technology and configured to hold a surgical microscope, in accordance with some embodiments.

FIG. 5 illustrates a surgical robotic system having multiple cameras constructed in accordance with some embodiments.

### DETAILED DESCRIPTION OF THE TECHNOLOGY

With reference now to the figures and several representative embodiments, the following detailed description is provided.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

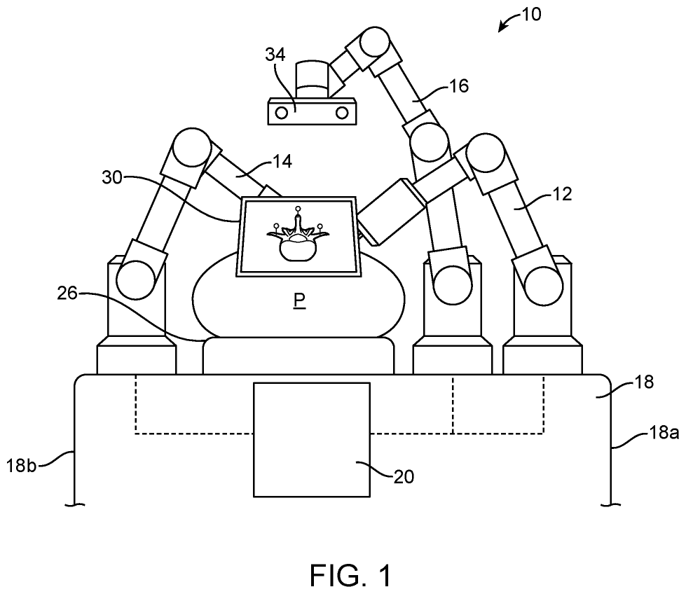

FIG. 1 shows a representative embodiment of a surgical robotic system 10 having first, second, and third surgical robotic arms 12, 14, and 16 mounted on a chassis 18. The chassis 18 typically comprises a single, rigid frame which provides a base or platform for the three surgical robotic arms 12, 14, and 16, where the surgical robotic arms are placed relatively far apart on the chassis on opposite longitudinal ends thereof, typically approximately one meter apart, thus allowing for desirable attributes such as reachability, maneuverability, and an ability to apply significant force. In the illustrated embodiment, surgical robotic arms 12 and 16 are on a first end 18a of the chassis 18 and surgical robotic arm 112 is on a second end 18b of the chassis. The chassis 18 may be mobile, e.g., being in the form of a mobile cart as described in commonly owned WO2022/195460, previously incorporated herein by reference. In other embodiments and implementations, the surgical arms 12, 14, and 16 can be mounted on a base or other structure of a surgical table. For control and manipulation of a display screen in accordance with the disclosed technology, the surgical robotic arms 12, 14, and 16 may be located on a stable platform that allows the arms to be moved within a common robotic coordinate system under the control of a surgical robotic controller, typically an on-board controller 20, typically have a user interface, such as a touch screen, a track pad, a mouse, a joystick, a roller ball, or the like (not shown).

A single, rigid chassis suitable for incorporation into the disclosed technology can comprise, consist of, or consist essentially of a single mobile cart, as disclosed for example in commonly owned PCT application no. PCT/IB2022/052297 (published as WO2022/195460), the full disclosure of which has been previously incorporated herein by reference. In other instances, however, the single, rigid chassis may comprise separate modules, platforms, or components, that are assembled at or near the surgical table, as described for example in commonly owned PCT Application PCT/EP2024/052353, entitled “Integrated Multi-Arm Mobile Surgical Robotic System,” filed on Jan. 29, 2024, the full disclosure of which is incorporated herein by reference. The single, rigid chassis may provide a stable base for all the surgical arms so that they may be accurately and precisely kinematically positioned and tracked by the surgical robotic controller in a single surgical robotic coordinate space.

The chassis 18 of the surgical robotic system 10 may be configured to be temporarily placed under a surgical table 26 when performing the robotic surgical procedure, allowing the first and third robotic surgical robotic arms 12 and 16 to be located on a first lateral side of the surgical table 26 and the second surgical robotic arm 14 to be located on a second lateral side. The robotic arms 12, 14, and 18 may optionally be configured to be retracted into the chassis 18 of the robotic surgical system, allowing the system to be moved into or out of the surgical field in a compact configuration.

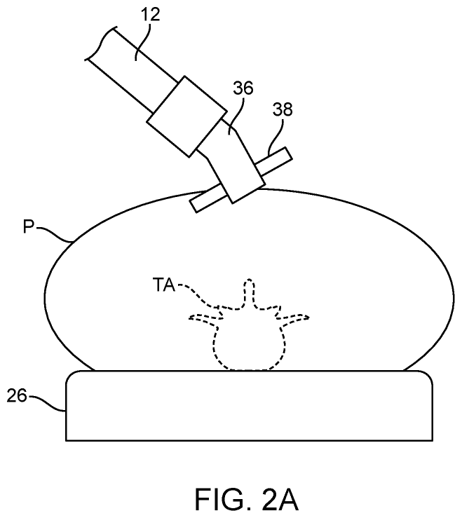

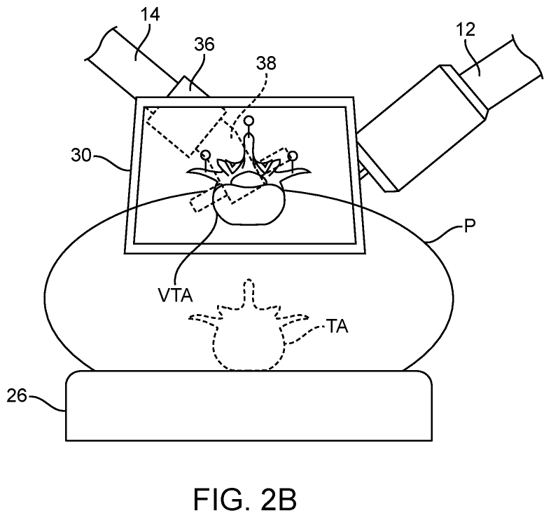

In accordance with the principles of the disclosed technologies, the first surgical robotic arm 12 can carry a display screen 30, the second surgical robotic arm 14 can carry a surgical tool, assembly, or other component (e.g., a tool 32 as shown in FIGS. 2A and 2B), and the third surgical robotic arm 34 can carry a three-dimensional navigation or other camera.

In some embodiments, the first surgical robotic arm 12 can be dedicated to carrying the display screen 30 and the display screen can be permanently (not interchangeably) affixed to a distal end of the arm 12. In other instances, the display can be interchangeably mounted on a “working” surgical robotic arm suitable for carry surgical tools, cameras, sensors, or the like and/or other but that would be less preferred.

The display screen 30 can be any conventional screen of the type used in surgical and/or general robotic applications. Often, the display screen 30 can be at least partly transparent so that an image or data can be presented in a “head up” format, commonly referred to as a head-up display (HUD). By placing a HUD along a user's line-of-sight (typically from a user's eyes to the surgical site when the user is located by the patient in a robotic surgery), the user can continue to directly view the surgical site without the need to look away.

Referring now to FIG. 2A, in the absence of a HUD or other display screen, a user can view the surgical robotic arm 12 which carries a surgical tool 36 in a gripper 36, but may not have “line-of sight” image or other information available regarding the location, size, orientation, or the like, of a target anatomy (shown in broken line as a vertebra) which may not be readily visible from an external location. Even in open surgical procedures, the view of a target anatomy can be obstructed, and in most minimally invasive procedures no direct view may exist.

Referring now to FIG. 2B, by placing display screen 30 in line-of sight with the tool 38 and the target anatomy, a virtual image of the target anatomy VTA can be presented on the screen in apparent alignment with the actual target anatomy TA. The robotic controller can determine the alignment of the virtual target anatomy VTA based upon a preoperative image or scan which has been provided to the robotic controller and registered to the surgical robotic coordinate space.

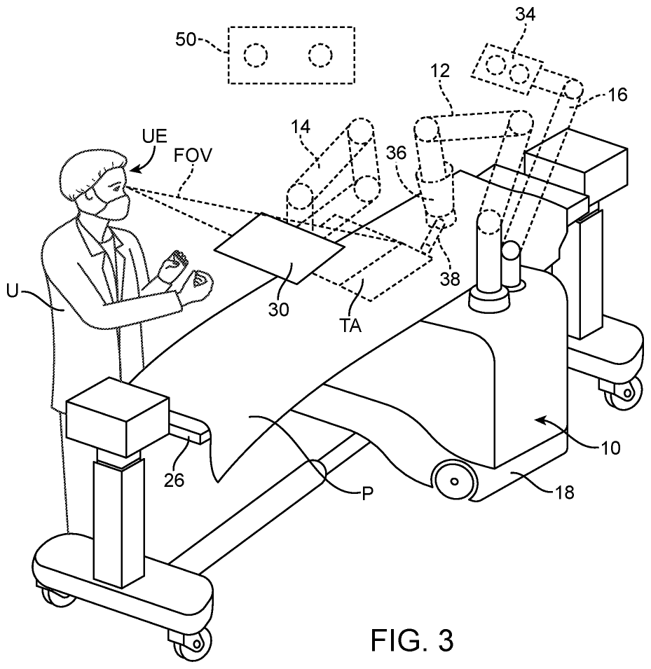

In some embodiments, the robotic controller 20 can automatically position and reposition the display screen 30 as the user moves about the surgical table and patient. As shown in FIG. 3, the controller 20 can optically track the user's eyes UE using, for example, either the on-board navigation camera 34 or a room-based camera 50 that can scan large parts of the surgery room. Once the location of the user's eyes UE is known within the surgical robotic coordinate space, the user's field-of-view FOV or line-of-sight can be calculated based upon the location of the target anatomy TA which is known based upon prior registration of the preoperative image in the surgical robotic coordinate space. The controller 20 can then position the display screen 30 along the field-of-view FOV or line-of-sight by kinematically positioning surgical robot arm 14 to locate the display screen along the line-of sight at a location which does not interfere with other robot functions, such as manipulation of the surgical tool by surgical robot arm 12, and/or the user's access to the robot and/or the patient.

A particular advantage is that the controller 20 is configured to kinematically and/or optically track the locations of all robotic components as well as tracking the patient P and the user U. Using such real-time locational information, the controller 20 can automatically adjust the positions of the display screen 30, the surgical tool 38, and/or the camera 34, as the user moves or changes her/his line-of-sight or the patient anatomy changes position.

The robotic controller 20 and display screen 30 of the disclosed technology may be configured to provide one or more virtual reality and/or augmented reality (VR/AR) elements or capabilities. In some embodiments, the display screens 30 can incorporate a virtual HUD technology, as just described. Other common augmented reality features include histochemical or other highlighting of target tissues, annotations showing target locations, and the like.

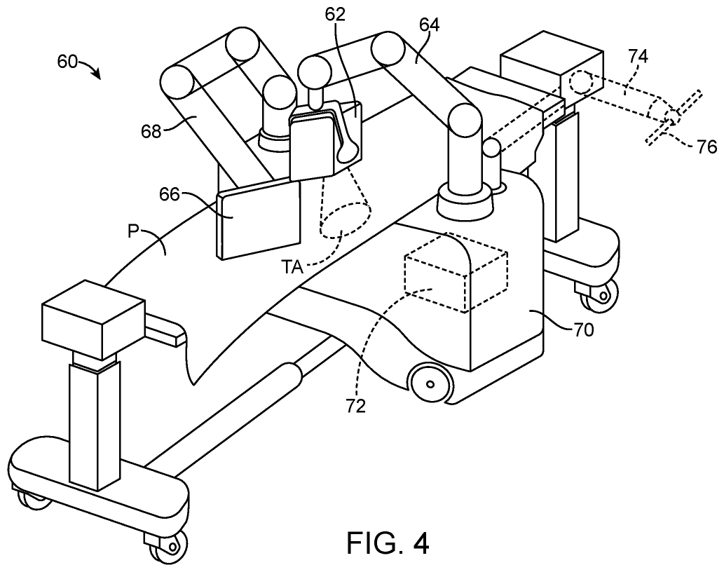

Referring now to FIG. 4, a surgical robot 60 according to the disclosed technology can be configured to carry a surgical microscope 62 on a first surgical robotic arm 64 and a display screen 66 on a second surgical robotic arm 68. The surgical robotic arms 64 and 66 can be mounted on a chassis and controlled by a robotic controller 72 as previously described with reference to surgical robotic system 10. In some embodiments, the surgical robot 60 can include at least one additional surgical robotic arm configured to carry a surgical tool, and additional surgical robotic arm(s), surgical tool(s), sensor(s), and the like can optionally be incorporated for performing specific robotic surgical procedures.

In some instances, the controller 72 can be configured to automatically reposition the display screen 66 as the user positions and repositions the microscope 62. The user may reposition the microscope 62 though an interface (not shown) causing the controller 72 to instruct the surgical arm 64 to move, as previously described, or in other instances the microscope and surgical robotic arm may be configured to be manually repositioned (with the user grasping and physically moving the microscope and robotic arm) with the controller kinematically and/or optically tracking the actual position in the surgical robotic coordinate space.

As with previously described embodiments, the controller 72 may automatically reposition the display screen 66 based upon any one or more of numerous criteria including but not limited to, convenience and “intuitiveness” of the alignment of the display screen with the microscope, the patient, and the target anatomy TA.

In some embodiments, the surgical robotic system may utilize active/robotic, rather than passive, virtual and/or augmented reality. The robotic arm holding the display screen 30 can be centrally coordinated with the robotic system from the control unit in the single chassis—in this way, the robotic arm (and, thus, the display screen) “knows where to go.” The display screen (which may or may not comprise a virtual and/or augmented reality element) may not be positioned by the user in the correct location in the surgical field but rather actively placed by the robotic system based on seeking out a marker or other feature/anatomical landmark with the assistance of onboard, coordinated surgical navigation. The movement may be coordinated based on the centrally coordinated robotic system knowing the patient and all robotic arms location and is able to synchronize all and the robotically deploy the virtual/augmented reality screen in the right place above the operated area.

In various representative embodiments involving a virtual and/or augmented reality screen, a navigation camera may optionally be integrated into the screen. The presence of the camera provides an additional feedback loop to the centrally coordinated robotic system by, for example, confirming that the robotic arm holding the virtual and/or augmented reality element has reached a desired position adjacent to an active or passive marker or a desired anatomical part/feature. The movement of the virtual and/or augmented reality screen (and, thus, the marker) is coordinated by the robotic system. This is distinct from currently available virtual and/or augmented reality systems that are navigation synchronized and passive where the user must bring the virtual and/or augmented reality element to the surgical field and a distant navigation camera provides guidance or that the user needs to wear a pair of goggles on his head which add discomfort.

Also, these goggles must be worn all the time even when this feature is not required. In some embodiments, the robotic arm can bring the virtual and/or augmented reality to the optimal location just in time when it is needed and then clear the way and not disturb the remainder of the surgical procedure.

Once the virtual and/or augmented reality screen with or without the integrated camera has been positioned adjacent to, for example, a marker or an anatomical feature, the user can take advantage of the virtual and/or augmented reality capabilities to, for example, enhance their view of anatomy that is difficult to visualize and/or not in their direct line of sight. Active coordination of the virtual and/or augmented reality element by the robotic system can confirm that it has been brought to the correct location and provides accuracy and predictability, along with opportunities for coordination with preoperative imaging and planning and also intra-operative imaging and guidance/navigation. This technique can allow, for example, to request the robotic system to position one robotic arm which holds the virtual/augmented reality screen perpendicularly to a tool that a second robotic arm is holding in relation to a desired location in the anatomical region. This can provide the user very valuable orienting visualization with minimum discomfort and unprecedented automation and efficiency.

In alternative embodiments, a virtual and/or augmented reality screen may facilitate another camera/sensor that detects the user's eyes/gaze. Accordingly, the robotic arm can actively position the screen not only in the optimal position and angulation towards the patient and relevant anatomy but also the optimal position and angulation towards the user, this represents a significant optimization of virtual and/or augmented reality in surgery while leaving the user free of any burden or hassle that is usually associated with the use of virtual and/or augmented reality technology.

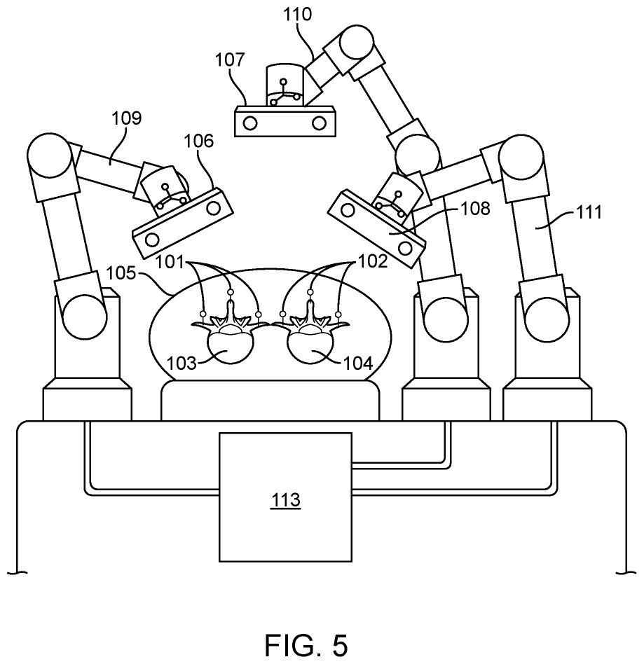

As shown in FIG. 5, multiple miniature markers 101, 102 (less than 5 cm in size and in some cases even smaller than 1 cm) may be placed on the relevant anatomy 103, 104 of a spinal surgery patient 105 during a surgical procedure by the physician. The miniature markers may optionally be placed with the assistance of preoperative imaging (e.g., CT or MRI), and additionally with the assistance of preoperative planning modalities. The markers may be active or passive and may optionally be placed on, for example, several aspects of several vertebrae in the patient's spine that requires surgical intervention.

The anatomy target(s) and markers can then be acquired and registered by intra-operative imaging (e.g., intraoperative CT). In this example, several robotic navigation cameras 106, 107, 108 are used that are, in turn, mounted on a corresponding number of robotic arms 109, 110, and 111 that are, affixed to a single chassis 112 with a control unit 113. Optionally, as described previously, a virtual and/or augmented reality screen may also be deployed using the said robotic arms. The control unit 113 coordinates the movement of the multiple robotic arms and/or the navigation cameras toward the anatomy target, creating a closed feedback loop. The use of multiple navigation cameras provides both redundancy and diversity of information about the anatomical targets of interest, which is crucial for accuracy and overall adequacy of information. The cameras may employ different technologies, for example infra-red and optical (RGB) modalities. The use of different modalities also provides diversity of information, thus increasing overall accuracy and quality of the provided information. The use of virtual and/or augmented reality provides clearer user visibility of anatomy elements that are out of the clear sight lines of the user.

A further robotic navigation camera may be mounted on a further robotic arm mounted to the same single chassis, wherein the further camera is positioned in an additional and supplementary distance and angulation from the surgical field (e.g., 10 cm to 50 cm), so that the whole surgical field may be imaged. Additional robotic arms may be disposed on the single chassis and may hold markers or end effectors. As all the robotic arms are disposed on the same chassis and their movement is coordinated within a common surgical robotic coordinate space by the control unit 113 contained in the chassis, one of skill in the art will realize that the movement of each of the various arms is related to the movement of the other robotic arms in a closed feedback loop. For example, if one of the robotic arms is holding a navigation camera and/or a virtual/augmented reality screen close to the desired anatomical region of interest (e.g., a particular vertebra) based on the position of a miniature marker, then the navigation camera held at a conventional distance can visualize the entire surgical field and assist in the placement of the other close-in navigation cameras adjacent to their anatomical regions of interest (e.g., adjacent vertebrae with markers already placed on them). This closed feedback loop can then be used to guide the deployment of a surgical tool and/or virtual/augmented reality screen that may be robotically brought to the surgical field as an end effector on a robotic arm.

The use of multiple navigation cameras and also, optionally, virtual and/or augmented reality screens, also enhances the quality of information by allowing for the collection of data pertaining to the projected shade or image of an object. If one navigation camera is imaging the anatomical target of interest from the optimal angulation to visualize, for example, a deep-seated tissue marker, a further camera positioned at a greater distance may be able to capture more information based on the projected image or shadow of the object of interest. Such enhanced visibility of deep-seated markers and anatomy features may also be provided by virtual and/or augmented reality screens which may be positioned in proximity of the operated area and while visualizing the situation to the user are also in parallel use their embedded cameras as part of the multi-cameras systems since this screen camera is mostly positioned above the operated area and in close proximity.

REFERENCE NUMBERS

| | |
|---|---|
| 10 | Surgical robotic system |
| 12, 14, 16 | Surgical robotic arms |
| 18 | Chassis |
| 18a/b | Longitudinal ends of chassis |
| 20 | Controller |
| 26 | Surgical table |
| 30 | Display screen |
| 32 | Surgical tool |
| 34 | Three-dimensional navigation camera |
| 36 | Gripper |
| 38 | Surgical tool |
| 40 | Target anatomy |
| 50 | Room camera |
| 60 | Surgical robot |
| 62 | Surgical microscope |
| 64, 68, 74 | Surgical robotic arm |
| 66 | Display screen |
| 70 | Chassis |
| 72 | Robotic controller |
| 76 | Surgical tool |
| 101, 102 | Miniature markers |
| 103, 104 | Patient anatomy (vertebra) |
| 10 | Surgical robotic system |
| 12, 14, 16 | Surgical robotic arms |
| 18 | Chassis |
| 18a/b | Longitudinal ends of chassis |
| 20 | Controller |
| 26 | Surgical table |
| 30 | Display screen |
| 32 | Surgical tool |
| 34 | Three-dimensional navigation camera or other imaging device |
| 36 | Gripper |
| 38 | Surgical tool |
| 40 | Target anatomy |
| 50 | Room camera |
| 60 | Surgical robot |
| 62 | Surgical microscope |
| 64, 68, 74 | Surgical robotic arm |
| 66 | Display screen |
| 70 | Chassis |
| 72 | Robotic controller |
| 76 | Surgical tool |
| 101, 102 | Miniature markers |
| 103, 104 | Patient anatomy (vertebra) |

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A surgical robotic system comprising:
a chassis;
a first surgical robotic arm mounted on the chassis and configured to carry an imaging device;
a second surgical robotic arms mounted on the chassis and configured to carry a display screen;
a third surgical robotic arm mounted on the chassis and configured to carry a surgical tool; and
a robotic controller;
wherein the control unit is configured to (1) control movement of the first surgical robotic arm to position the imaging sensor to view a target location on a patient anatomy, (2) control movement of the second surgical robotic arm to orient the display screen in a predetermined spatial relationship with the target location on the patient anatomy, and (3) display an image of the patient's anatomy on the anatomical display screen.

2. The surgical robotic system of claim 1, wherein the image of the patient's anatomy at least partly comprises a preoperative image.

3. The surgical robotic system of claim 1, wherein the image of the patient's anatomy at least partly comprises a real-time image.

4. The surgical robotic system of claim 1, wherein the predetermined spatial relationship comprises locating the anatomical display screen along a user's line-of-sight.

5. The surgical robotic system of claim 4, wherein the display screen is at least partly transparent and configured to allow a user to view an image on the display while maintaining a line-of-sight view of the target anatomy through the display screen.

6. The surgical robotic system of claim 1, wherein the predetermined relationship comprises locating the display screen over the target anatomy and the anatomical display image comprises internal structures not externally visible.

7. The surgical robotic system of claim 6, wherein the display screen is located over the surgical tool, allowing a user to align the tool with a target internal anatomical structure visible on the anatomical display image.

8. The surgical robotic system of claim 1, wherein the imaging device is a surgical microscope and the controller is configured to display an output of the surgical microscope on the display screen while the display screen and the target location are in the user's line-of-site.

9. The surgical robotic system of claim 1, wherein the controller is configured to (1) at least in part automatically control the first and second robot surgical arms and (2) allow a user to at least in part control the third surgical robotic arm in real-time.

10. The surgical robotic system of claim 1, wherein the display screen includes an additional imaging device.

11. The surgical robotic system of claim 10, wherein the controller is configured to align the display screen with the patient anatomy based at least in part on an output of the additional image sensor

12. The surgical robotic system of claim 1, wherein the controller is further configured to receive position information for the user's eyes.

13. The surgical robotic system of claim 1, wherein the controller is further configured to determine a line-of-sight from the position of the user's eyes to the target location.

14. A method for performing robotic surgery on a patient, said method comprising:
controlling a first surgical robotic arm to position an imaging sensor to scan a target surgical site on a patient anatomy;
controlling a second surgical robotic arm to orient a display screen in a predetermined relationship with the target surgical site on the patient anatomy; and
controlling a third surgical robotic arm to position a surgical tool to be used in performing the robotic surgery;
wherein a user is positioned adjacent to the patient at a location which allows direct viewing of the display screen and the target location and wherein the first and second surgical robotic arms are at least in part automatically controlled by a robotic controller and the third surgical robotic arm is at least in part controlled by real-time input from the user to the controller as the user views the display and the patient anatomy.

15. The method of claim 14, further comprising displaying a preoperative image on the display screen.

16. The method of claim 14, further comprising displaying a real-time image on the display screen.

17. The method of claim 14, wherein the display screen is at least partly transparent allowing the user to view an image on the display while maintaining a line-of-sight view of the surgical site through the display screen.

18. The method of claim 14, wherein the robotic controller controls at least the first surgical robotic arm to align the display screen along a line-of-sight from the user's eyes to the surgical site on the patient's anatomy.

19. The method of claim 14, wherein further comprising scanning the user with an imaging device to determine a position of the user's eyes.

20. The method of claim 14, wherein the imaging device is the imaging device carried by the first surgical robotic arm.

21. The method of claim 14, wherein the imaging sensor comprises a surgical microscope and the robotic controller delivers an image from the microscope to the display screen and positions the display screen in line-of-sight with the patient anatomy being viewed by the surgical microscope.

22. A robotically coordinated robotic virtual and/or augmented reality system comprising:
at least two robotic arms mounted on a single chassis incorporating a central control unit configured to control the movement of the robotic arms; at least one surgical navigation camera held by one of the at least two robotic arms; and
at least one virtual and/or augmented reality element held by one of the at least two robotic arms;
wherein the system is configured such that the central control unit directs placement of the virtual and/or augmented reality element into an optimal position for enhanced visualization of relevant anatomy by a user.

23. The system of claim 22, wherein the at least two robotic arms are three robotic arms.

24. The system of claim 23, wherein two of the robotic arms hold navigation cameras and one of the robotic arms holds a virtual and/or augmented reality screen.

25. The system of claim 24, wherein one of the navigation cameras is held at a close distance to the anatomy of interest and one of the navigation cameras is held at a further distance from the anatomy of interest.

26. The system of claim 25, wherein the virtual and/or augmented reality screen is held in an optimal position to enhance visibility of anatomy that is out of the user's direct line of sight.

27. The system of claim 22, wherein the virtual and/or augmented reality screen is actively placed by the robotically coordinated system in an optimal position for enhancing user visibility without interfering with other navigation elements.

28. The system of claim 22, wherein the virtual and/or augmented reality element incorporates an additional navigation camera.

29. The system of claim 22, wherein the at least two robotic arms are four robotic arms.

30. The system of claim 29, wherein two of the robotic arms hold navigation cameras and one of the robotic arms holds a virtual and/or augmented reality screen and one of the robotic arms holds a surgical tool.

31. The system of claim 30, wherein one of the navigation cameras is held at a close distance to the anatomy of interest and one of the navigation cameras is held at a further distance from the anatomy of interest.

32. The system of claim 30, wherein the virtual and/or augmented reality screen is held in an optimal position to enhance visibility of anatomy that is out of the user's direct line of sight.

33. The system of claim 30, wherein the virtual and/or augmented reality screen is actively placed by the robotically coordinated system in an optimal position for enhancing user visibility without interfering with other navigation elements or surgical elements.

34. The system of claim 30, wherein the virtual and/or augmented reality element incorporates an additional navigation camera.

35. The system of claim 30, wherein the surgical tool is moved into the surgical field by the robotically coordinated system with additional information provided by the virtual and/or augmented reality screen.
